(19) United States
(12) Patent Application Publication
(10) Pub. No.: US 2022/0411857 A1
(43) Pub. Date: Dec. 29, 2022

(54) COMPOSITIONS AND METHODS OF NUCLEIC ACID AMPLIFICATION

(71) Applicant: Enumerix, Inc., Palo Alto, CA (US)

(72) Inventors: Hei Mun Christina FAN, Palo Alto, CA (US); Eleen Yee Lam SHUM, San Carlos, CA (US)

(21) Appl. No.: 17/836,736

(22) Filed: Jun. 9, 2022

Related U.S. Application Data
(63) Continuation of application No. PCT/US2020/064119, filed on Dec. 9, 2020.
(60) Provisional application No. 62/946,371, filed on Dec. 10, 2019.

(51) Int. Cl. C12Q 1/6848

(52) U.S. Cl. CPC C12Q 1/6848; C12Q 1/6818

(57) ABSTRACT

Described herein are methods for amplifying and detecting a specific nucleic acid in a nucleic acid sample. The methods may comprise amplifying a target nucleic acid and detecting the target nucleic acid in the presence of a fluorescent probe and a probe complement. Also described herein are probe complements for detecting specific nucleic acid sequences with reduced background fluorescence.

### CROSS-REFERENCE

This application is a continuation application of PCT Patent Application number PCT/US2020/064119, filed Dec. 9, 2020, which claims priority to U.S. Provisional Application No. 62/946,371, filed Dec. 10, 2019, each of which is incorporated by reference herein in its entirety.

### BACKGROUND

Fluorescent probes are often used in detecting specific nucleic acid sequence. Fluorescent nucleic acid probes, such as fluorescent hydrolysis probes, have been widely used in real-time PCR and digital PCR assays. Fluorescent probes, including hydrolysis probes, emit background fluorescence that may prevent reliable data collection, particularly in three dimensional samples, including thick samples or samples with significant depth. There is a need for fluorescent probes with reduced background fluorescence for imaging samples in three dimensions.

### SUMMARY

In various aspects, the present disclosure provides a method of amplifying a nucleic acid molecule in a sample, comprising: contacting a nucleic acid molecule in a biological sample to a polymerase, a first fluorescent probe comprising a first probe nucleic acid that anneals to a target genetic sequence, and a first probe complement comprising a first complementary nucleic acid that anneals to the first probe nucleic acid; and amplifying the nucleic acid molecule to form a population of amplified molecules.

In some aspects, the method further comprises imaging the population of amplified molecules. In some aspects, the method further comprises analyzing the imaging. In some aspects, the imaging is light sheet imaging. In some aspects, the analyzing comprises assessing fluorescence in the population of amplified molecules. In some aspects, a sequence of the first complementary nucleic acid lacks a string of guanines. In some aspects, the string of guanines is at least 4 or more sequential guanines in the sequence.

In various aspects, the present disclosure provides a method for determining the ratio of a target genetic sequence in a population of nucleic acid molecules, comprising: diluting the population of nucleic acid molecules from a sample to form a set comprising a plurality of assay samples; amplifying the nucleic acid molecules within the assay samples to form a population of amplified molecules in the assay samples of the set; analyzing the amplified molecules in the assay samples of the set to determine a first number of assay samples comprising the target genetic sequence and a second number of assay samples comprising a reference genetic sequence; comparing the first number to the second number to ascertain a ratio which reflects the composition of the sample.

In some aspects, about 10% of the plurality of assay samples comprises zero or one nucleic acid molecules. In some aspects, analyzing the amplified molecules comprises fluorescence imaging of the amplified molecules. In some aspects, the method comprises adding a first fluorescent probe comprising a first probe nucleic acid that anneals to the target genetic sequence, a second fluorescent probe comprising a second probe nucleic acid that anneals to the reference genetic sequence, a first probe complement comprising a first complementary nucleic acid that anneals to the first probe nucleic acid, and a second probe complement that comprises a second complementary nucleic acid that anneals to the second probe nucleic acid. In some aspects, imaging the amplified molecules comprises light sheet imaging. In some aspects, a sequence of the first probe complementary nucleic acid, a sequence of the second probe complementary nucleic acid, or both, lack a string of guanines. In some aspects, the string of guanines is at least 4 or more sequential guanines.

In various aspects, the present disclosure provides a method for reducing background fluorescence in a nucleic acid amplification assay, the method comprising contacting a sample with: a fluorescent probe comprising a fluorescent moiety conjugated to a probe nucleic acid, wherein the probe nucleic acid is configured to anneal to a target nucleic acid; a probe complement comprising a quenching moiety conjugated to a complementary nucleic acid, wherein the complementary nucleic acid is substantially complementary to a region of the probe nucleic acid, and wherein the quenching moiety has an absorption spectrum that overlaps with an emission spectrum of the fluorescent moiety; and a nucleic acid polymerase capable of hydrolyzing the probe nucleic acid when the probe nucleic acid is annealed to the target nucleic acid.

In some aspects, the region of the probe nucleic acid is the full length of the probe nucleic acid. In some aspects, the region of the probe nucleic acid is less than the full length of the probe nucleic acid. In some aspects, the complementary nucleic acid is fully complementary to the region of the probe nucleic acid. In some aspects, the complementary nucleic acid comprises at least one base pair mismatch with respect to the region of the probe nucleic acid. In some aspects, the quenching moiety of the complementary nucleic acid is proximal to the fluorescent moiety of the probe nucleic acid. In some aspects, proximal is the quenching moiety of the complementary nucleic acid is 5 or less nucleotide bases away from the fluorescent moiety of the probe nucleic acid. In some aspects, proximal is the quenching moiety of the complementary nucleic acid is opposite to the fluorescent moiety of the probe nucleic acid. In some aspects, proximal is the quenching moiety of the complementary nucleic acid is 1 nucleotide base away from the fluorescent moiety of the probe nucleic acid.

In some aspects, a melting temperature of the complementary nucleic acid and the region of the probe nucleic acid is from 30° C. to 60° C. In some aspects, the complementary nucleic acid anneals to the probe nucleic acid at a temperature of about 20° C. to about 25° C. In some aspects, a melting temperature of the complementary nucleic acid and the region of the probe nucleic acid is lower than a melting temperature of the probe nucleic acid and the target nucleic acid. In some aspects, the quenching moiety quenches the fluorescent moiety when the complementary nucleic acid is annealed to the probe nucleic acid. In some aspects, the complementary nucleic acid lacks a string of guanines. In some aspects, the string of guanines is at least 4 or more sequential guanines in the sequence.

In various aspects, the present disclosure provides a probe complement comprising a quenching moiety conjugated to a complementary nucleic acid, wherein the quenching moiety has an absorption spectrum that overlaps with an emission spectrum of a fluorescent moiety, and wherein the complementary nucleic acid is substantially complementary to a probe nucleic acid conjugated to the fluorescent moiety.

In some aspects, the region of the probe nucleic acid is the full length of the probe nucleic acid. In some aspects, the region of the probe nucleic acid is less than the full length of the probe nucleic acid. In some aspects, the complementary nucleic acid is fully complementary to the region of the probe nucleic acid. In some aspects, the complementary nucleic acid comprises at least one base pair mismatch with respect to the region of the probe nucleic acid. In some aspects, a melting temperature of the complementary nucleic acid and the region of the probe nucleic acid is from 30° C. to 60° C. In some aspects, the complementary nucleic acid anneals to the probe nucleic acid at a temperature of about 20° C. to about 25° C. In some aspects, the quenching moiety quenches the fluorescent moiety when the complementary nucleic acid is annealed to the probe nucleic acid. In some aspects, a sequence of the probe complement lacks a string of guanines. In some aspects, the string of guanines is at least 4 or more sequential guanines in the sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B shows cross-sectional fluorescence images of three-dimensional samples of fluorescent droplets labeled with fluorescent probes comprising a nucleic acid having a 5′ fluorophore and a 3′ quencher for use in digital polymerase chain reactions. Digital polymerase chain reaction (dPCR) was performed in droplets in each tube and imaged using light sheet imaging. FIG. 1A shows two representative cross-sections of a tube imaged using a first exemplary fluorescent probe. FIG. 1B shows two representative cross-sections of a tube imaged using a second exemplary fluorescent probe. Both exemplary fluorescent probes exhibit background fluorescence although the first exemplary fluorescent probe in FIG. 1A has lower background fluorescence than the second exemplary fluorescent probe in FIG. 1B.

FIGS. 2A-2B shows schematics of exemplary probe complement (“cProbe”) configurations. The cProbe comprise a 3′ quencher and anneals to a fluorescent probe comprising a nucleic acid and a fluorophore. Optionally, the fluorescent probe comprises a quencher at the opposite end of the nucleic acid from the fluorophore. FIG. 2A shows a cProbe configuration comprising a 3′ quencher and a nucleic acid sequence that is complementary except for a mismatch to the fluorescent probe nucleic acid. Annealing of the cProbe to the fluorescent probe brings the cProbe 3′ quencher in close proximity to the 5′ fluorophore of the fluorescent probe, resulting in quenching of the fluorescence emission of the 5′ fluorophore of the fluorescent probe. FIG. 2B shows a cProbe configuration comprising a 3′ quencher and a nucleic acid sequence that is complementary to a segment of the 5′ region of the fluorescent probe nucleic acid. Annealing of the cProbe to the fluorescent probe brings the 3′ quencher of the cProbe in close proximity to the 5′ fluorophore of the fluorescent probe, resulting in quenching the fluorescence emission of the 5′ fluorophore of the fluorescent probe.

FIGS. 3A-3B shows cross-sectional fluorescence images of three-dimensional samples of fluorescent droplets labeled with fluorescent probes comprising a nucleic acid having a 5′ fluorophore and a 3′ quencher. Digital PCR (dPCR) was performed in droplets in each tube an imaged using light sheet imaging. FIG. 3A shows two representative cross-sections of a tube imaged using a fluorescent probe without a cProbe. FIG. 3B shows two representative cross-sections of a tube imaged using a fluorescent probe and a cProbe. Addition of the cProbe greatly reduced the background fluorescence of the three-dimensional sample compared to the sample dPCR without the cProbe.

### DETAILED DESCRIPTION

Fluorescent imaging may be used to detect and quantify the presence of a specific nucleic acid molecule in a sample. Fluorescent probes comprising a nucleic acid sequence that anneals to the target nucleic acid may be used to detect the specific nucleic acid molecule. Fluorescent probes, such as TaqMan probes, that are hydrolyzed upon binding to the target nucleic acid, thereby increasing probe fluorescence, are frequently used to detect the specific nucleic acid molecule. Fluorescent probes, such as Light Cycler probes, that comprise two fluorescent nucleic acids that bind to the target nucleic acid thereby increasing probe fluorescence via Förster resonance energy transfer (FRET), may also be used to detect the specific nucleic acid molecule. Detection of a specific nucleic acid molecule using fluorescence may be performed using assays such as digital polymerase chain reaction (dPCR), digital isothermal amplification (e.g., digital LAMP or digital helicase dependent amplification or digital recombinase polymerase amplification), or hybridization assays (e.g., nanoballs with rolling circle amplification). Fluorescent detection of nucleic acid samples (e.g., digital PCR, digital isothermal amplification, or hybridization assays) may be performed in a variety of media or sample formats. In some embodiments, fluorescent detection of nucleic acid molecules of samples may be performed in droplets, microwells, nanowells, gel substrates, or solid substrates. In some embodiments, fluorescent detection is performed by imaging, which may be one-dimensional (1D), two-dimensional (2D), or three-dimensional (3D) imaging. In some embodiments, 3D imaging is performed by light sheet microscopy. The sample may be a three-dimensional (3D) sample, such as a sample in a tube or vial. The sample may be a two-dimensional (2D) sample, such as a sample in a planar array or plate. The sample may be a one-dimensional (1D) sample, such as a sample with individual droplet or molecules passing through a flow channel.

### Methods of Nucleic Acid Amplification

Described herein are methods for amplifying and detecting a specific nucleic acid in a nucleic acid sample. In some embodiments, the methods described herein may be used to determine a population ratio of a target nucleic acid and a reference nucleic acid. A method for amplifying a specific nucleic acid in a nucleic acid sample may comprise diluting a biological sample comprising nucleic acid template molecules to form a set comprising a plurality of assay samples. For example, a biological sample may comprise chromosomal DNA from a human subject. In some embodiments, diluting the template molecules (e.g., nucleic acid molecules comprising a target nucleic acid) may comprise partitioning the template molecules. For example, the template molecules may be partitioned into droplets, microwells, or nanowells. Alternatively, the template molecule and its amplified products can also be localized in space (e.g., by restricted diffusion inside a gel matrix, or anchored onto a surface) such that partitioning of a template molecule and its amplified products does not require any physical barriers.

Each partition may comprise either one copy of the template molecule or zero copies of the template molecule. In some embodiments, a dilution factor is selected so that at least 95%, at least 97.5%, at least 98%, at least 99%, at least 99.5%, or at least 99.9% of the partitions comprise no more than one copy of the template molecule. In some embodiments, a dilution factor is selected so that 2%, 3%, 4%, 5%, 6%, 7%, 8%, 9%, 10%, 15%, 20%, 30%, 40%, 50%, 60%, 70% 80%, 90%, 95%, 98%, 99%, 99.9% or any percentage therebetween of the partitions comprise no more than one copy of the template molecule. In some embodiments, a dilution factor is selected so that about 2%, about 3%, about 4%, about 5%, about 6%, about 7%, about 8%, about 9%, or about 10% of the partitions comprise no more than one copy of the template molecule. Each partition may comprise either one copy or zero copies of the reference nucleic acid molecule. In some embodiments, a dilution factor is selected so that at least 95%, at least 97.5%, at least 98%, at least 99%, at least 99.5%, or at least 99.9% of the partitions comprise no more than one copy of the reference nucleic acid. In some embodiments, a dilution factor is selected so that 2%, 3%, 4%, 5%, 6%, 7%, 8%, 9%, 10%, 15%, 20%, 30%, 40%, 50%, 60%, 70% 80%, 90%, 95%, 98%, 99%, 99.9% or any percentage therebetween of the partitions comprise no more than one copy of the reference nucleic acid. In some embodiments, a dilution factor is selected so that about 2%, about 3%, about 4%, about 5%, about 6%, about 7%, about 8%, about 9%, or about 10% of the partitions comprise no more than one copy of the reference nucleic acid. Each partition may comprise either one copy or zero copies of the target nucleic acid. In some embodiments, a dilution factor is selected so that at least 95%, at least 97.5%, at least 98%, at least 99%, at least 99.5%, or at least 99.9% of the partitions comprise no more than one copy of the target nucleic acid. In some embodiments, a dilution factor is selected so that 2%, 3%, 4%, 5%, 6%, 7%, 8%, 9%, 10%, 15%, 20%, 30%, 40%, 50%, 60%, 70% 80%, 90%, 95%, 98%, 99%, 99.9% or any percentage therebetween of the partitions comprise no more than one copy of the target nucleic acid. In some embodiments, a dilution factor is selected so that about 2%, about 3%, about 4%, about 5%, about 6%, about 7%, about 8%, about 9%, or about 10% of the partitions comprise no more than one copy of the target nucleic acid. In some embodiments, a dilution factor is selected so that no more than 5%, no more than 2.5%, no more than 2%, no more than 1%, no more than 0.5%, or no more than 0.1% of the partitions comprise both a reference nucleic acid and a target nucleic acid.

The template molecules may be amplified within the assay samples to form a population of amplified molecules in the assay samples of the set. In some embodiments, amplification is performed by polymerase chain reaction (PCR), digital PCR (dPCR), isothermal amplification (e.g., loop-mediated isothermal amplification (LAMP) or helicase dependent amplification), or digital isothermal amplification. In some embodiments, an amplification reaction may comprise a polymerase, optionally with nuclease activity, for example a DNA polymerase. In some embodiments, the amplification reaction may comprise a fluorescent probe, such as a fluorescent nucleic acid probe. In some embodiments, the fluorescent nucleic acid probe may be a hydrolysis probe.

The fluorescent probe may comprise a probe nucleic acid that anneals to a target nucleic acid and a fluorescent moiety conjugated to and at the end of the probe nucleic acid. The fluorescent probe may comprise a probe nucleic acid that anneals to a reference nucleic acid and a fluorescent moiety conjugated to and at the end of the probe nucleic acid. The fluorescent moiety may be an organic fluorophore (e.g., an Alexa-fluor, a Cy dye, a HEX fluorophore, a FAM fluorophore, VIC, TEX615, SUN, Yakima Yellow, ATTO dye, TYE dye, TET, JOE, ROX, TAMRA, BODIPY dye, IRDye, or a rhodamine). In some embodiments, the fluorescent probe may further comprise a quenching moiety conjugated to the opposite end of the probe nucleic acid. The quenching moiety may suppress the fluorescence of the fluorescent moiety through fluorescence resonance energy transfer (FRET). The quenching moiety may have an absorption spectrum that overlaps the emission spectrum of the fluorescent moiety. In some embodiments, the quenching moiety may comprise a dark quencher (examples—Black Hole quenchers, Iowa Black FQ, Iowa Black RQ, Dabysl, Qxl quenchers, IRDye QC-1). In some embodiments, the quenching moiety of the complementary nucleic acid is proximal to the fluorescent moiety of the probe nucleic acid. In some embodiments, proximal is the quenching moiety of the complementary nucleic acid is 5 or less nucleotide bases away from the fluorescent moiety of the probe nucleic acid. In some embodiments, proximal is the quenching moiety of the complementary nucleic acid is opposite to the fluorescent moiety of the probe nucleic acid. In some embodiments, proximal is the quenching moiety of the complementary nucleic acid is 1 nucleotide base away from the fluorescent moiety of the probe nucleic acid.

The amplification reaction may comprise a probe complement. The probe complement may suppress the fluorescence of the fluorescent probe that is not bound to the target nucleic acid or the reference nucleic acid. The probe complement may suppress the fluorescence of the fluorescent probe that has not been hydrolyzed by the polymerase with nuclease activity. The probe complement may comprise a complementary nucleic acid that is substantially complementary to the probe nucleic acid or a region of the probe nucleic acid and a quenching moiety conjugated to one end of the complementary nucleic acid. The sequence of the probe complement (e.g., the sequence of the complementary nucleic acid) may lack strings of guanines. Strings of guanines may prevent the suppression of the background fluorescence. In a first exemplary configuration, the complementary nucleic acid comprises a mismatch relative to the probe nucleic acid. In a second exemplary configuration, the complementary nucleic acid is fully complementary to a segment of the probe nucleic acid. The complementary nucleic acid may comprise 1, 2, 3, 4, 5, or more mismatches relative to the probe nucleic acid. The complementary nucleic acid may anneal to a segment of the probe nucleic acid that is from 1 to 10, from 5 to 15, from 10 to 20, from 15 to 25, from 20 to 30, from 25 to 35, from 30 to 40 nucleic acids long. The complementary nucleic acid may be from 1 to 10, from 5 to 15, from 10 to 20, from 15 to 25, from 20 to 30, from 25 to 35, from 30 to 40 nucleic acids long. The probe nucleic acid may be from 1 to 10, from 5 to 15, from 10 to 20, from 15 to 25, from 20 to 30, from 25 to 35, from 30 to 40 nucleic acids long. In some embodiments, the sequence of the probe complement lacks 4 or more sequential guanines. In further embodiments, the sequence of the probe complement lacks 4, 5, 6, 7, 8, 9 10, 20, 30, or 40, or any number therebetween, of sequential guanines. In some embodiments, the sequence of the probe complement lacks 4 sequential guanines.

The quenching moiety may have an absorption spectrum that overlaps the emission spectrum of the fluorescent moiety. In some embodiments, the quenching moiety is selected to increase the spectral overlap between the absorption spectrum of the quenching moiety and the emission spectrum of the fluorescent moiety. In some embodiments, the fluorescent moiety is selected to increase the spectral overlap between the absorption spectrum of the quenching moiety and the emission spectrum of the fluorescent moiety. The quenching moiety and the fluorescent moiety may be conjugated to opposite ends of their respective nucleic acids. For example, the quenching moiety may be conjugated to the 3′ end of the complementary nucleic acid, and the fluorescent moiety may be conjugated to the 5′ end of the probe nucleic acid. The quenching moiety may be conjugated to the 5′ end of the complementary nucleic acid, and the fluorescent moiety may be conjugated to the 3′ end of the probe nucleic acid.

The complementary nucleic acid sequence may be designed to anneal the probe nucleic acid at an assay temperature, for example a temperature at which imaging may be performed. In some embodiments, the complementary nucleic acid may anneal the probe nucleic acid at room temperature. The complementary nucleic acid may anneal the probe nucleic acid at a temperature of about 15° C., about 16° C., about 17° C., about 18° C., about 19° C., about 20° C., about 21° C., about 22° C., about 23° C., about 24° C., or about 25° C. The complementary nucleic acid may have a melting temperature with respect to the fluorescent probe nucleic acid sequence of between about 30° C. and about 60° C. The complementary nucleic acid may have a melting temperature with respect to the fluorescent probe nucleic acid sequence of from 30° C. to 50° C., from 30° C. to 40° C., from 40° C. to 50° C., from 45° C. to 55° C., from 50° C. to 60° C., from 55° C. to 65° C., or from 60° C. to 70° C. In some embodiments, a melting temperature of the complementary nucleic acid and the region of the probe nucleic acid is lower than a melting temperature of the probe nucleic acid and the target nucleic acid. In some embodiments, the complementary nucleic acid does not anneal to the probe nucleic acid at an annealing temperature of a PCR reaction.

The method for amplifying the specific nucleic acid in a nucleic acid sample may comprise analyzing the amplified molecules in the assay samples of the set to determine a first number of assay samples which contain the selected sequence (e.g., a target nucleic acid) and a second number of assay samples which contain a reference genetic sequence (e.g., a reference nucleic acid). Analyzing the amplified molecules may comprise imaging the amplified molecules. In some embodiments, the amplified molecules may be imaged by fluorescence imaging. For example, the amplified molecules may be imaged by detecting the fluorescence of the fluorescent probe, as described herein (e.g., a fluorescent nucleic acid probe). Imaging may be performed using one-dimensional, two-dimensional, or three-dimensional imaging methods, as described herein. For example, amplified molecules in a three-dimensional sample (e.g., a tube comprising droplets (e.g., aqueous droplets formed in immiscible fluids, such as hydrocarbon oil, silicon oil, fluorinated oil, etc.) may be imaged using light sheet imaging. In some embodiments, imaging may be performed in the presence of a probe complement. In some embodiments, fluorescence imaging may be performed at two wavelengths. For example, fluorescence imaging may be performed at a first wavelength to measure the fluorescence of a fluorescent probe that anneals to a target nucleic acid, and fluorescence imaging may be performed at a second wavelength to measure the fluorescence of a fluorescent probe that anneals to a reference nucleic acid.

The method for amplifying the specific nucleic acid in a nucleic acid sample may comprise comparing the first number (e.g., corresponding to a target nucleic acid) to the second number (e.g., corresponding to a reference nucleic acid) to ascertain a ratio which reflects the composition of the biological sample. For example, the number of partitions with fluorescence of a fluorescent probe that anneals to a target nucleic acid may be compared to the number of partitions with the fluorescence of a fluorescent probe that anneals to a reference nucleic acid.

The probe complements described herein may be used in combination with a fluorescent probe. In some embodiments, the probe complements may be used in combination with a fluorescent nucleic acid probe. In some embodiments, the fluorescent nucleic acid probe may be a hydrolysis probe or a Förster resonance energy (FRET) probe.

The probe complements described herein may be used in one-dimensional, two-dimensional, or three-dimensional imaging techniques. For example, the probe complements may be used in epi-fluorescence microscopy, total internal reflection fluorescence (TIRF) microscopy, fluorescence illumination and detection with a camera or photodiode, light sheet imaging, confocal microscopy, or imaging droplets passing through a flow cell channel.

The probe complements described herein may be used in assays such as digital polymerase chain reaction (dPCR), digital isothermal amplification (e.g., digital LAMP or digital helicase dependent amplification or digital recombinase polymerase amplification), or hybridization assays (e.g., nanoballs with rolling circle amplification). The probe complements described herein may be added to samples in droplets, microwells, nanowells, gel substrates, or solid substrates.

### Fluorescent Imaging of Nucleic Acid Samples

To quantify or detect the nucleic acid sample, the sample may be imaged. In some embodiments, the sample may be imaged following the digital PCR, digital isothermal amplification, or hybridization assay. The imaging technique may be selected based on the sample format. For example, a one-dimensional (1D) sample (e.g., a flow cell) may be imaged using a one-dimensional imaging technique. One-dimensional imaging techniques may include epi-fluorescence microscopy, total internal reflection fluorescence (TIRF) microscopy, or fluorescence illumination and detection with a camera or photodiode (e.g., a gel fluorescence imager). In some embodiments, a one-dimensional (1D) sample is imaged by the passing of a droplet or a fluorescent nucleic acid molecule through a flow cell channel, e.g., passing of a droplet or a fluorescent nucleic acid molecule of the sample one by one, and detecting fluorescence. A two-dimensional (2D) sample (e.g., a planar array, a microwell plate, or a nanowell plate) may be imaged using a two-dimensional imaging technique. Two-dimensional imaging techniques may include epi-fluorescence microscopy, total internal reflection fluorescence (TIRF) microscopy, or fluorescence illumination and detection with a camera (e.g., a gel fluorescence imager). A three-dimensional sample (e.g., a tube or a cell) may be imaged using a three-dimensional imaging technique. Three-dimensional imaging techniques may include light sheet imaging, or confocal microscopy. Three-dimensional imaging technique may comprise illuminating and imaging sequential cross-sections of the three-dimensional sample to produce a three-dimensional image data set.

In each fluorescent imaging technique, there is a need to improve the signal to noise ratio to increase signal detection accuracy and precision. One way to improve the signal to noise ratio is to reduce background fluorescence. This is particularly important in three-dimensional imaging techniques since three-dimensional imaging may be imaged through a thick sample or bulk solution, compounding the effects of high fluorescence background. For example, in a digital sample comprising PCR compartments (e.g., droplets) packed together, any residual fluorescence in neighboring negative compartments may compound and interfere with the detection of the positive compartment. Thus, in 3D visualization, it is preferable to suppress background fluorescence as much as possible.

### Systems and Methods for Background Fluorescence Reduction

The methods for amplifying and detecting a specific nucleic acid in a nucleic acid sample performed in the presence of probe complements may have reduced background fluorescence compared to methods performed in the absence of probe complements. The methods performed in the presence of probe complements may have increased signal to noise ratio compared to methods performed in the absence of probe complements. Nucleic acid probes comprising a nucleic acid that anneals to a target nucleic acid, a fluorophore conjugated to one end of the nucleic acid, and a quencher conjugated to the other end of the nucleic acid may be used to reduce fluorescence background. Such probes may function by being hydrolyzed upon annealing to the target nucleic acid, thereby separating the fluorophore from the quencher. In solution, when the probe is not bound to the target nucleic acid, the quencher may suppress the fluorescence of the fluorophore, reducing background fluorescence. However, such background fluorescence reduction may not be sufficient for three-dimensional imaging. In some embodiments, the background fluorescence may be further reduced by designing a hairpin sequence into the nucleic acid sequence of the probe to bring the quencher closer to the fluorophore when in solution. However, hairpin structures may be difficult to design for a desired target nucleic acid sequence.

Nucleic acid probes comprising a first nucleic acid that anneals to a target nucleic acid conjugated to a first fluorophore conjugated and a second nucleic that anneals to the target nucleic acid near the first nucleic acid conjugated to a second fluorophore that forms a FRET pair with the first fluorophore may be used to reduce fluorescence background. Such probes may function by FRET transfer from the first fluorophore to the second fluorophore upon binding of the first nucleic acid and the second nucleic acid to the target nucleic acid. In solution, when the probe is not bound to the target nucleic acid, FRET may be low, reducing background fluorescence. However, such background fluorescence reduction may not be sufficient for three-dimensional imaging.

Described herein are methods, systems, and kits to reduce background fluorescence in fluorescent nucleic acid detection imaging. A system to reduce background fluorescence may comprise a fluorescent probe and a probe complement. The fluorescent probe may comprise a fluorescent probe as described herein. The probe complement may comprise a probe complement as described herein. The probe complement may reduce the fluorescence of a fluorescent probe that is not bound to a target nucleic acid. The probe complement may reduce the fluorescence of a fluorescent probe that has not been hydrolyzed by a DNA polymerase with nuclease activity. The probe complement may reduce the fluorescence of a fluorescent probe by annealing to the fluorescent probe and quenching the fluorescence of the fluorophore.

### Kits

A kit for amplification and detection of a nucleic acid sample may comprise a probe complement, as described herein, and a fluorescent probe, as described herein. A kit for amplification and detection of a nucleic acid sample may comprise a DNA polymerase. A kit for amplification and detection of a nucleic acid sample comprising a probe complement may provide reduced background fluorescence as compared to a kit for amplification and detection of a nucleic acid sample that does not comprise a probe complement.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### EXAMPLES

The following examples are illustrative and non-limiting to the scope of the devices, systems, fluidic devices, kits, and methods described herein.

### Example 1

### Light Sheet Imaging of Digital PCR Samples Using Hydrolysis Probes

This example describes light sheet imaging of digital PCR (dPCR) samples using hydrolysis probes. Digital PCR was performed in droplets in PCR tubes. Briefly, a nucleic acid sample was partitioned into droplets such that each droplet most likely contained either zero or one nucleic acid copy, based on Poisson statistics. A polymerase chain reaction (PCR) was performed on the droplets in the presence of a DNA polymerase having nuclease activity and a fluorescent hydrolysis probe (TaqMan hydrolysis probe) complementary to a segment of the nucleic acid sample being amplified. Upon binding of the hydrolysis probe to the amplified nucleic acid, the DNA polymerase hydrolyzed the nucleic acid sequence of the hydrolysis probe, thereby separating the quencher from the fluorophore. Hydrolysis probes that did not anneal to a nucleic acid had suppressed fluorescence due to the proximity of the quencher to the fluorophore. The samples were imaged in the PCR tubes using light sheet imaging. Digital PCR was performed using either a hydrolysis probe having high fluorescence background or a hydrolysis probe having low fluorescence background.

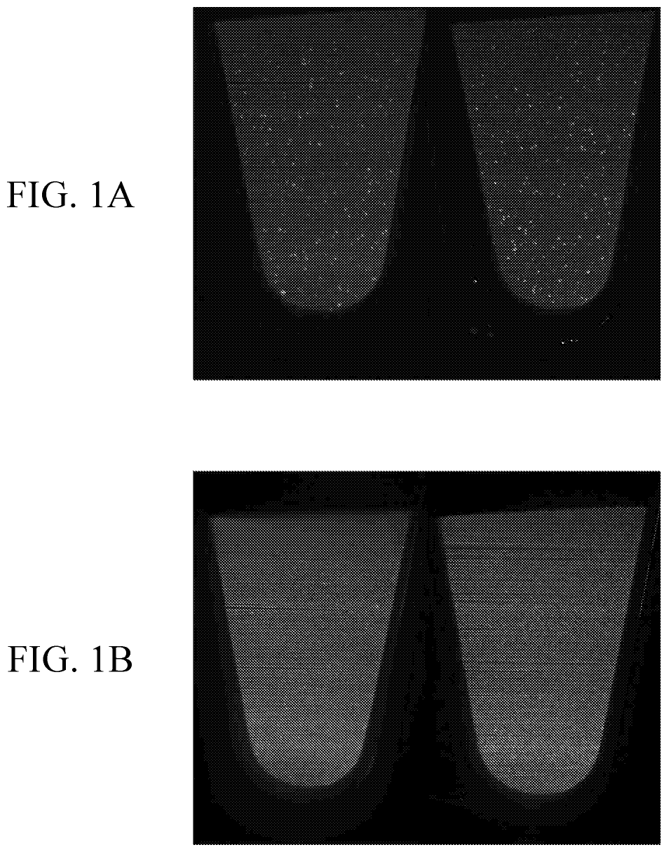

FIGS. 1A-1B show cross-sectional fluorescence images of three-dimensional samples of fluorescent droplets labeled with fluorescent hydrolysis probes (e.g. TaqMan hydrolysis probes). Digital polymerase chain reaction (dPCR) was performed in droplets in each tube an imaged using light sheet imaging. FIG. 1A shows two representative cross-sections of a tube imaged using a hydrolysis probe having low fluorescence background. FIG. 1B shows two representative cross-sections of a tube imaged using a hydrolysis probe having high fluorescence background.

The sequences of the nucleic acids of the hydrolysis probes were analyzed to determine a mechanism for the reduction of fluorescence background. Hydrolysis probes comprising a short nucleic acid sequence with a fluorophore at the 5′ end and a quencher at the 3′ end generally exhibited low fluorescent background if the nucleic acid sequence formed a hairpin structure with a negative change in Gibbs free energy (ΔG) (as in the probe used in FIG. 1A). The hairpin structure may bring the quencher closer to the fluorophore, thereby suppressing the probe background.

### Example 2

### Design of Probe Complements for Reduced Background Fluorescence

This example describes the design of probe complements for reduced background fluorescence. Probe complements were designed for use in digital PCR amplification reactions. Probe complements were designed to reduce the background fluorescence of nucleic probes, such as hydrolysis probes. The probe complements comprised a quencher located at the end of a nucleic acid sequence. The nucleic acid sequence was designed to be substantially or fully complementary to all or part of the nucleic acid sequence of the fluorescent nucleic acid probe.

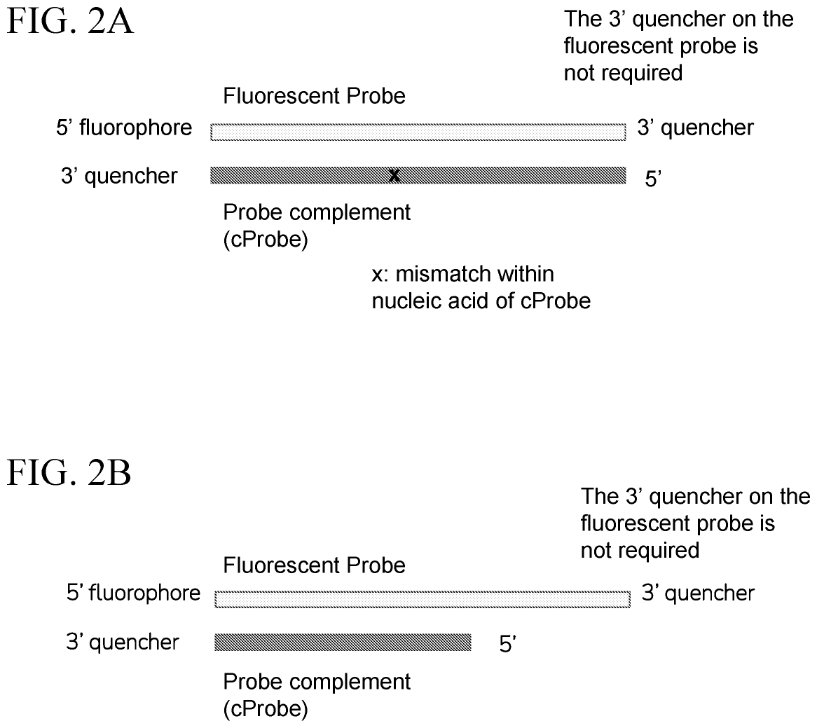

FIGS. 2A-2B shows schematics of exemplary probe complement configurations. The probe complement anneals to a fluorescent probe comprising a nucleic acid and a fluorophore. Optionally, the fluorescent probe comprises a quencher at the opposite end of the nucleic acid from the fluorophore.

In a first probe complement configuration, the quencher was positioned at the 3′ end of a nucleic acid sequence that was near complementary to a nucleic acid sequence of a fluorescent probe having a fluorophore on at the 5′ end of the nucleic acid sequence. FIG. 2A shows a probe complement configuration with a probe complement comprising a nucleic acid sequence that is near complementary to the fluorescent probe nucleic acid. Annealing of the probe complement to the fluorescent probe brings a quencher in close proximity to the fluorophore, quenching the fluorescence emission.

In a second probe complement configuration, the quencher was positioned at the 3′ end of a nucleic acid sequence that was fully complementary to a segment of the nucleic acid sequence of the fluorescent probe having a fluorophore on at the 5′ end of the nucleic acid sequence. FIG. 2B shows probe complement configuration with a probe complement comprising a nucleic acid sequence that is fully complementary to a segment of the fluorescent probe nucleic acid. Annealing of the probe complement to the fluorescent probe brings a quencher in close proximity to the fluorophore, quenching the fluorescence emission.

In both configurations, the probe complement nucleic acid sequence was designed so that it annealed to the fluorescent probe nucleic acid sequence at the temperature at which imaging was done. Imaging, as performed in EXAMPLE 1, was performed at room temperature (approximately 21° C. to 25° C.). The probe complement nucleic acid sequence was designed to have a melting temperature with respect to the fluorescent probe nucleic acid sequence between 30° C. and 60° C. such that the probe complement did not anneal to the fluorescent probe at the PCR annealing temperature. Melting temperatures of the probe complement nucleic acid sequence relative to the fluorescent probe nucleic acid sequence were easier to predict for fully complementary sequences (as illustrated in FIG. 2B) than for sequences containing mismatches (as illustrated in FIG. 2A), as predicted melting temperatures for mismatched sequences varied based on the melting temperature algorithm used.

### Example 3

### Identification of Trisomy 21 Using Digital PCR with Probe Complements

This example describes the identification of trisomy 21 using digital PCR with probe complement quenchers. Digital PCR was performed as described in EXAMPLE 1 on human nucleic acid samples using a hydrolysis probe with locked nucleic acids (TaqMan locked nucleic acid (LNA) probes) with 10 bases targeted to either chromosome 21(“chr21”) or chromosome 18 (“chr18”). The hydrolysis probe targeted to chromosome 21 had a HEX fluorophore at the 5′ end of the LNA sequence and a black hole quencher at the 3′ end of the LNA sequence. The hydrolysis probe targeted to chromosome 18 had a FAM fluorophore at the 5′ end of the LNA sequence and a black hole quencher at the 3′ end of the LNA sequence.

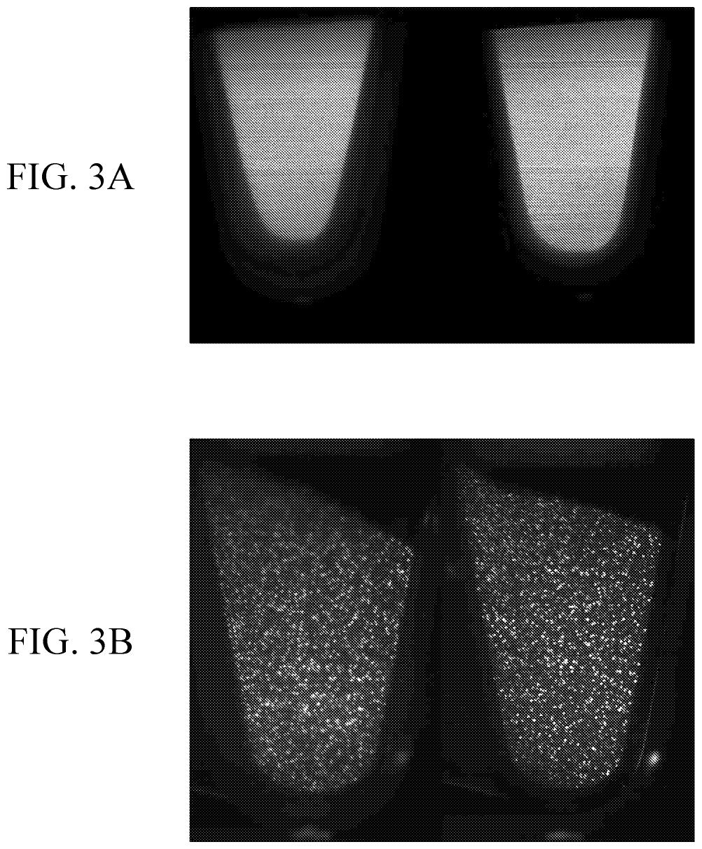

In a first assay, background fluorescence was compared between two samples. In the first sample, digital PCR was performed in the absence of probe complements. In the second sample, two probe complements, one that annealed to the chromosome 21 fluorescent probe and one that annealed to the chromosome 18 fluorescent probe, were included in the digital PCR reaction. FIG. 3A shows two representative cross-sections of a tube imaged using a hydrolysis probe without a probe complement. FIG. 3B shows two representative cross-sections of a tube imaged using a hydrolysis probe with a probe complement. Addition of the probe complement reduced the background fluorescence of the three-dimensional sample.

In a second assay, digital PCR was performed on samples as before in the presence of the two probe complements targeted to the chromosome 21 fluorescent probe and the chromosome 18 fluorescent probe. Each sample contained chromosomal DNA from a human with trisomy 21 or control sample with the normal number of chromosomes. Samples were imaged using light sheet imaging and fluorescent particles corresponding to chromosome 18 and chromosome 21 were counted in each sample. The resulting counts are shown in TABLE 1. Assay sensitivity was sufficient to identify trisomy 21 samples based on the ratio of chromosome 21 fluorescent particle counts to chromosome 18 fluorescent particle counts. As expected, the trisomy 21 samples had a chromosome 21 to chromosome 18 ratio (“chr2l:chr18”) of about 1.5, while the control samples had a chromosome 21 to chromosome 18 ratio of about 1.

TABLE 1
Fluorescent counts of chromosome 18 and chromosome
21 in trisomy 21 and control samples

| | Green | Blue | | |
|---|---|---|---|---|
| | Count | Count | Measured | Expected |
| Sample | (chr21) | (chr18) | chr21:chr18 | chr21:chr18 |
| T21 NA04616 (trisomy) | 7422 | 4850 | 1.53 | 1.5 |
| T21 NA04965 (trisomy) | 35863 | 24213 | 1.48 | 1.5 |
| Mother (control) | 6139 | 5884 | 1.04 | 1.0 |
| Son #2 (control) | 4802 | 4665 | 1.03 | 1.0 |

In a third assay, a trisomy 21 nucleic acid sample was diluted in 2-fold serial dilutions. Digital PCR was performed on each dilution in the presence of hydrolysis probes for chromosome 21 and chromosome 18 and quencher probes targeted to the chromosome 21 and chromosome 18 fluorescent probes, as previously described. Digital PCR was also performed on a control sample without a nucleic acid template (“NTC”). At higher sample concentrations (low titration series numbers), the assay accurately detected the chromosome 21 to chromosome 18 ratio (“chr2l:chr18”). Accuracy decreased at lower sample concentrations (high titration series numbers) due to Poisson statistics at low copy numbers. The fluorescent counts and chromosome 21 to chromosome 18 ratios for each sample of the titration series are shown in TABLE 2.

TABLE 2
Titration series of genomic DNA from a trisomy 21 individual

| | Expected | Measured | Measured | |
|---|---|---|---|---|
| Titration | Blue Count | Blue Count | Green Count | Measured |
| Series | (chr18) | (chr18) | (chr21) | chr21:chr18 |
| 1 | 200k | 105354 | 152310 | 1.50 |
| 2 | 100k | 72182 | 104334 | 1.48 |
| 3 | 39375 | 32364 | 48465 | 1.5 |
| 4 | 19687 | 17889 | 27302 | 1.53 |
| 5 | 4922 | 6433 | 10051 | 1.56 |
| 6 | 1231 | 1608 | 2548 | 1.58 |
| 7 | 307 | 387 | 573 | 1.48 |
| 8 | 77 | 88 | 122 | 1.39 |
| 9 | 19 | 22 | 25 | 1.14 |
| NTC | 0 | 7 | 0 | 0 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method, comprising:
generating a three-dimensional distribution of fluorescent droplets within a tube, wherein the three-dimensional distribution of fluorescent droplets comprises aqueous droplets packed within an immiscible fluid, and
wherein the three-dimensional distribution exhibits reduced background fluorescence upon:
within a droplet of the three-dimensional distribution, contacting a nucleic acid molecule to a polymerase, a first fluorescent probe comprising a first probe nucleic acid that anneals to a target genetic sequence, and a first probe complement comprising a first complementary nucleic acid that anneals to the first probe nucleic acid;
amplifying the nucleic acid molecule to form a population of amplified molecules; and
imaging the tube containing the droplet, with a 3D imaging technique.

2. The method of claim 1, wherein the 3D imaging technique comprises light sheet imaging.

3-5. (canceled)

6. The method of claim 1, wherein a sequence of the first complementary nucleic acid lacks a string of at least four sequential guanines.

7-8. (canceled)

9. The method of claim 1, wherein at least 10% of droplets of the three-dimensional distribution of fluorescent droplets comprise zero or one nucleic acid molecules.

10. (canceled)

11. The method of claim 1, VOL further comprising, for a second droplet of the three-dimensional distribution of fluorescent droplets, adding the first fluorescent probe comprising the first probe nucleic acid that anneals to the target genetic sequence, a second fluorescent probe comprising a second probe nucleic acid that anneals to a reference genetic sequence, the first probe complement comprising the first complementary nucleic acid that anneals to the first probe nucleic acid, and a second probe complement that comprises a second complementary nucleic acid that anneals to the second probe nucleic acid.

12. (canceled)

13. The method of claim 11, wherein a sequence of the first probe complementary nucleic acid, a sequence of the second complementary nucleic acid, or both, lack a string of four or more sequential guanines.

14. (canceled)

15. A method for reducing background fluorescence in a nucleic acid amplification assay, the method comprising contacting a sample with:
a fluorescent probe comprising a fluorescent moiety conjugated to a probe nucleic acid, wherein the probe nucleic acid is configured to anneal to a target nucleic acid; and
a probe complement comprising a quenching moiety conjugated to a complementary nucleic acid, wherein the complementary nucleic acid is substantially complementary to a region of the probe nucleic acid, and wherein the quenching moiety has an absorption spectrum that overlaps with an emission spectrum of the fluorescent moiety.

16. (canceled)

17. The method of claim 15, wherein the region of the probe nucleic acid is less than the full length of the probe nucleic acid.

18. (canceled)

19. The method of claim 15, wherein the complementary nucleic acid comprises at least one base pair mismatch with respect to the region of the probe nucleic acid.

20. (canceled)

21. The method of claim 20, wherein the quenching moiety of the complementary nucleic acid is 5 or fewer base pairs away from the fluorescent moiety of the probe nucleic acid.

22-23. (canceled)

24. The method of claim 15, wherein a melting temperature of the complementary nucleic acid and the region of the probe nucleic acid is from 30° C. to 70° C.

25. (canceled)

26. The method of claim 15, wherein a melting temperature of the complementary nucleic acid and the region of the probe nucleic acid is lower than a melting temperature of the probe nucleic acid and the target nucleic acid.

27. The method of claim 15, wherein the quenching moiety quenches the fluorescent moiety when the complementary nucleic acid is annealed to the probe nucleic acid.

28. The method of claim 15, wherein the complementary nucleic acid lacks a string of four or more sequential guanines.

29. (canceled)

30. A probe complement comprising a quenching moiety conjugated to a complementary nucleic acid, wherein the quenching moiety has an absorption spectrum that overlaps with an emission spectrum of a fluorescent moiety, and wherein the complementary nucleic acid is substantially complementary to a probe nucleic acid conjugated to the fluorescent moiety.

31. (canceled)

32. The probe complement of claim 30, wherein the region of the probe nucleic acid is less than the full length of the probe nucleic acid.

33. (canceled)

34. The probe complement of claim 30, wherein the complementary nucleic acid and the region of the probe nucleic acid comprise at least one base pair mismatch when the complementary nucleic acid and the region of the probe nucleic acid are optimally bound.

35. The probe complement of claim 30, wherein a melting temperature of the complementary nucleic acid and the region of the probe nucleic acid is from 30° C. to 70° C.

36. (canceled)

37. The probe complement of claim 30, wherein the quenching moiety quenches the fluorescent moiety when the complementary nucleic acid is annealed to the probe nucleic acid.

38. The probe complement of claim 30, wherein a sequence of the probe complement lacks a string of four or more sequential guanines.

39. (canceled)
